(19) United States
(12) Patent Application Publication
(10) Pub. No.: US 2011/0217289 A1
(43) Pub. Date: Sep. 8, 2011

(54) Melt-Coated Dosage Forms

(75) Inventors: Karl Kolter, Limburgerhof (DE); Dejan Djuric, Mannheim (DE)

(73) Assignee: BASF SE, Ludwigshafen (DE)

(21) Appl. No.: 13/040,534

(22) Filed: Mar. 4, 2011

Related U.S. Application Data
(60) Provisional application No. 61/310,731, filed on Mar. 5, 2010.

(51) Int. Cl. A61K 39/395; A61K 9/50; B05D 3/00; A61K 31/216; A61K 31/495; A61K 31/5415; A61K 31/4164; A61K 31/496; A61P 43/00

(52) U.S. Cl. 424/130.1; 424/490; 424/497; 427/2.16; 514/543; 514/255.04; 514/226.5; 514/396; 514/254.07

(57) ABSTRACT

Formulations of sparingly water-soluble active ingredientscomprising carrier particles provided with active ingredient-containing coatings, the sparingly soluble active ingredients being embedded in coatings composed of amphiphilic copolymers, and the coatings being applied in the form of a solvent-free melt.

### CROSS-REFERENCE TO RELATED APPLICATIONS

This application claims priority under 35 U.S.C. § 119(e) to U.S. patent application Ser. No. 61/310,731, filed Mar. 5, 2010, which is hereby incorporated by reference in its entirety.

### TECHNICAL FIELD

The present invention relates to carrier particles coated with active ingredient-containing coatings, wherein a sparingly water-soluble active ingredient is embedded in a coating composed of amphiphilic copolymers which are obtained by polymerizing vinyl acetate and N-vinyllactams in the presence of a polyether, and the coatings are applied in the form of a melt. The invention further relates to processes for producing such coated carrier particles and agglomerates of such particles, and to the use thereof in pharmaceutical administration forms.

### BACKGROUND

The coating of particles with active ingredient-containing coatings is effected typically by spray application of solutions of the coating materials.

A disadvantage of processing from solution is the complicated handling of organic solvents, which places high demands on the environment and equipment. Moreover, it is frequently difficult to find an appropriate organic solvent for sparingly soluble active ingredients.

WO 2005/034908 discloses applying melts or dispersions which comprise a combination of fibrates and statins as active ingredients to carrier particles in a fluidized bed granulator. The active ingredient combinations are in the form of solid solutions or solid dispersions of the active ingredients in hydrophilic or hydrophobic or pH-dependently soluble polymers. The granules thus obtained can be pressed to tablets.

Amphiphilic copolymers such as graft polymers, obtained by free-radical polymerization of vinyl acetate and N-vinyllactams in the presence of a polyether are known per se.

WO 2007/051743 discloses the use of water-soluble or water-dispersible copolymers of N-vinyllactam, vinyl acetate and polyethers as solubilizers for pharmaceutical, cosmetic, nutritional, agrochemical or other industrial uses. It is stated quite generally therein that the corresponding graft polymers can also be processed with the active ingredients in the melt.

WO 2009/013202 discloses that such graft polymers of N-vinyllactam, vinyl acetate and polyethers can be melted in an extruder and mixed with pulverulent or liquid active ingredients and processed to tablets.

There is a need to find an improved process for coating and agglomerating carrier particles.

### SUMMARY

Accordingly, a process has been developed for coating carrier particles with active ingredient-containing coatings, wherein a sparingly water-soluble active ingredient is embedded in a coating composed of amphiphilic copolymers which are obtained by polymerizing vinyl acetate and N-vinyllactams in the presence of a polyether, wherein the coatings are applied in the form of a melt.

The melt is solvent-free. In the context of the present invention, “solvent-free” means that less than 5000 ppm of solvent is present.

In addition, correspondingly coated carrier particles have been found.

Suitable amphiphilic copolymers for embedding of water-soluble active ingredients and the processibility thereof by melt extrusion are known, for example, from WO 2007/051743.

Corresponding copolymers are obtained by free-radically initiated polymerization of a mixture of
i) 30 to 80% by weight of N-vinyllactam,
ii) 10 to 50% by weight of vinyl acetate and
iii) 10 to 50% by weight of a polyether,
with the proviso that the sum of i), ii) and iii) is 100% by weight.

In one embodiment of the invention, preferred copolymers obtained from:
i) 30 to 70% by weight of N-vinyllactam
ii) 15 to 35% by weight of vinyl acetate and
iii) 10 to 35% by weight of a polyether are used.

Copolymers used with particular preference are obtainable from:
i) 40 to 60% by weight of N-vinyllactam
ii) 15 to 35% by weight of vinyl acetate and
iii) 10 to 30% by weight of a polyether.

Copolymers used with very particular preference are obtainable from:
i) 50 to 60% by weight of N-vinyllactam
ii) 25 to 35% by weight of vinyl acetate and
iii) 10 to 20% by weight of a polyether.

For the preferred and particularly preferred compositions too, the proviso applies that the sum of components i), ii), and iii) is 100% by weight.

Useful N-vinyllactams are N-vinylcaprolactam or N-vinylpyrrolidone, or mixtures thereof. Preference is given to using N-vinylcaprolactam.

Accordingly, an amphiphilic copolymer formed from N-vinylcaprolactam, vinyl acetate and a polyether is especially preferred.

The graft bases used are polyethers. Useful polyethers are preferably polyalkylene glycols. The polyalkylene glycols may have molecular weights of 1000 to 100 000 Da [daltons], preferably 1500 to 35 000 Da, more preferably 1500 to 10 000 Da. The molecular weights are determined proceeding from the OH number measured to DIN 53240.

Particularly preferred polyalkylene glycols are polyethylene glycols. Additionally suitable are also polypropylene glycols, polytetrahydrofurans or polybutylene glycols which are obtained from 2-ethyloxirane or 2,3-dimethyloxirane.

Suitable polyethers are also random or block copolymers of polyalkylene glycols obtained from ethylene oxide, propylene oxide and butylene oxides, for example polyethylene glycol-polypropylene glycol block copolymers. The block copolymers may be of the AB or of the ABA type.

The preferred polyalkylene glycols also include those which are alkylated on one or both OH end groups. Useful alkyl radicals include branched or unbranched C₁- to C₂₂-alkyl radicals, preferably C₁-C₁₈-alkyl radicals, for example methyl, ethyl, n-butyl, isobutyl, pentyl, hexyl, octyl, nonyl, decyl, dodecyl, tridecyl or octadecyl radicals.

General processes for preparing the copolymers used in accordance with the invention are known per se. The preparation is effected by free-radically initiated polymerization, preferably in solution, in nonaqueous organic solvents or in mixed nonaqueous/aqueous solvents. Suitable preparation processes are described, for example, in WO 2007/051743 and WO 2009/013202, explicit reference being made to the disclosure thereof with regard to the preparation process.

The amphiphilic copolymers used are especially polymers which are obtained by polymerizing vinyl acetate and N-vinyllactams in the presence of a polyether.

It is also possible for further polymers or low molecular weight substances to be incorporated into the coating.

The process according to the invention is notable in that carrier particles are coated with a melt. The melt is composed of sparingly soluble active ingredient, solubilizing polymer and optionally further additives. Cooling and solidification of the melt result in a coating which comprises the active ingredient in dissolved form.

The coating of the carrier particles with a melt can be effected in customary fluidized bed units. In this case, the melt is sprayed onto a fluidized bed composed of carrier particles.

The melt is advantageously already provided, prior to the actual spraying, in a temperature-controlled reservoir vessel. In one embodiment of the invention, the melt consisting of polymer, active ingredient and optionally further additives can be sprayed by means of nozzles with the aid of a likewise temperature-controllable melt pump. The nozzles used may be one-substance nozzles or multisubstance nozzles. Suitable multisubstance nozzles are especially two-substance nozzles.

In addition to the fluidized bed units, it is also possible to use other equipment in which the carrier particles are set in motion by rotation of tanks or by incoming air, for example coating tanks, intensive mixers, horizontal drum coaters, Kugelcoaters, Innojet units. Also suitable for the application of the formulations described is what is known as the jet fluidized bed (Procell technology).

The feed air temperatures are typically between 30 and 200° C., preferably between 40 and 120° C. The product temperatures are generally between 20 and 100 and preferably between 30 and 80° C.

In a further embodiment of the invention, the melt can also be applied without atomization, for example by pouring on in a thin stream.

As already described, the melt is solvent-free. The total content of solvents typically used for such purposes, such as alkanols, for example ethanol, methanol or isopropanol, and also acetone, ethyl acetate, dichloromethane, chloroform, dimethylformamide and/or methyl ethyl ketone, should be less than 5000 ppm. The content of alkanols should preferably be less than 10 ppm.

Suitable carrier particles are particularly spherical or at least approximately spherical particles, known as “nonpareils”. In one embodiment of the invention, the nonpareils are entirely, i.e. to an extent of 100% by weight, of pharmaceutical excipients. The nonpareils may comprise customary pharmaceutical excipients, for example sucrose, carrageenan, starch or microcrystalline cellulose. They are available in different sizes (100-2000 μm). The particle sizes are typically 700-1000 μm.

The carrier particles used may also be tablets or granules.

Typically, the weight ratio of active ingredient to amphiphilic polymer in the coating is between 1:99 and 80:20, preferably between 10:90 and 60:40. This results, in the final solidified polymer coating of the carrier particles, in active ingredient concentrations of 1 to 80% by weight, based on the total mass.

It is also possible to coat granules or fine powders as carrier particles, though the latter case can also result in agglomeration of the carrier particles. In one embodiment, such granules or powders may also comprise one or more active ingredients.

The layer thickness of the active ingredient-containing coatings of amphiphilic copolymer may be 5 to 1000 and preferably 10 to 700 μm.

According to the invention, the term “sparingly water-soluble” also comprises virtually insoluble substances and means that, for a solution of the substance in water at 20° C., at least 30 to 100 g of water is required per g of substance. In the case of virtually insoluble substances, at least 10 000 g of water are required per g of substance.

In the context of the present invention, sparingly-water soluble substances are preferably understood to mean biologically active substances such as active pharmaceutical ingredients for humans and animals, active cosmetic or agrochemical ingredients, or food supplements or active dietetic ingredients.

In addition, useful sparingly soluble substances to be solubilized also include dyes such as inorganic or organic pigments.

According to the invention, useful biologically active substances include, in principle, all solid active ingredients which have a melting point below the decomposition point under the melting conditions of the coating mixture. The copolymers can generally be processed at temperatures up to 260° C. The lower temperature limit is guided by the composition of the mixtures to be melted and the sparingly soluble substances to be processed in each case. The lower temperature limit may be 30° C.

The lower temperature depends on the viscosity of the melt, which must be low enough to be sprayed. In addition, the nozzle shapes and diameters are of significance for the melt viscosity, which is to be established via the temperature.

The active pharmaceutical ingredients used are substances with water solubility ranging from insoluble to sparingly soluble. According to DAB 9 (Deutsches Arzneimittelbuch, German Pharmacopeia), the solubility of active pharmaceutical ingredients is classified as follows: sparingly soluble (soluble in 30 to 100 parts of solvent); slightly soluble (soluble in 100 to 1000 parts of solvent); virtually insoluble (soluble in more than 10 000 parts of solvent). The active ingredients may come from any indication sector.

Examples here include benzodiazepines, antihypertensives, vitamins, cytostatics—especially taxol, anesthetics, neuroleptics, antidepressives, antivirals, for example anti-HIV drugs, antibiotics, antimycotics, antidementives, fungicides, chemotherapeutics, urologics, thrombocyte aggregation inhibitors, sulfonamides, spasmolytics, hormones, immunoglobulins, sera, thyroid therapeutics, psychopharmaceuticals, Parkinson's drugs and other antihyperkinetics, ophthalmics, neuropathy preparations, calcium metabolism regulators, muscle relaxants, anesthetics, lipid-lowering drugs, liver therapeutics, coronary drugs, cardiac drugs, immunotherapeutics, regulatory peptides and inhibitors thereof, hypnotics, sedatives, gynaecologicals, gout remedies, fibrinolytics, enzyme preparations and transport proteins, enzyme inhibitors, emetics, blood-flow stimulators, diuretics, diagnostics, corticoids, cholinergics, biliary therapeutics, antiasthmatics, bronchodilators, beta-receptor blockers, calcium antagonists, ACE inhibitors, arteriosclerotic drugs, anti-inflammation drugs, anticoagulants, antihypotensives, antihypoglycemics, antihypertensives, antifibrinolytics, antiepileptics, antiemetics, antidotes, antidiabetics, antiarrhythmics, antianemics, antiallergics, anthelmintics, analgesics, analeptics, aldosterone antagonists, slimming drugs.

It is also possible to process sparingly soluble active ingredients from traditional Chinese medicine.

The content of inventive solubilizer in the pharmaceutical formulation is, depending on the active ingredient, in the range from 20 to 99% by weight.

It is of course possible to add further pharmaceutically customary excipients to the inventive formulation, for example further solubilizers, polymers, dyes, inorganic carriers, disintegrants, gel formers, retardants, antioxidants, aromas, plasticizers, buffer substances. The incorporation of gastric juice-resistant polymers or of retarding polymers allows the release of the active ingredient to be controlled.

The addition of crystallization-inhibiting substances, for example Kollidon 30, allows the stability of the solid solutions to be increased.

Examples of suitable plasticizers include triacetin, triethyl citrate, glyceryl monostearate, polyethylene glycols or poloxamers.

Suitable additional solubilizers are interface-active substances with an HLB (Hydrophilic Lipophilic Balance) value greater than 11, for example hydrogenated castor oil ethoxylated with 40 ethylene oxide units (Cremophor® RH 40), castor oil ethoxylated with 35 ethylene oxide units (Cremophor EL), Polysorbate 80, poloxamers or sodium laurylsulfate.

Dyes are, for example, iron oxides, titanium dioxide, triphenylmethane dyes, azo dyes, quinoline dyes, indigotin dyes, carotenoids, in order to dye the administration forms, opacifiers, such as titanium dioxide or talc, in order to increase the transparency and to save dyes.

In addition to use in cosmetics and pharmacy, the formulations produced in accordance with the invention are also suitable for use in the foods sector, for example, for the incorporation of sparingly water-soluble or water-insoluble nutrients, assistants or additives, for example, fat-soluble vitamins or carotenoids.

The use of the formulations obtained in accordance with the invention in agrochemistry may comprise, inter alia, formulations which comprise pesticides, herbicides, fungicides or insecticides, and in particular also those formulations of crop protection compositions which are used as formulations for spraying or watering.

The coated carrier particles obtained in accordance with the invention can be used as capsule fillings or in sachets.

The coated carrier particles in the form of pellets or granules can also be pressed to tablets. It is also possible to use further customary tableting aids such as fillers, binders, disintegrants, and also flow aids and separating aids.

With the aid of the process according to the invention, it is possible to obtain active ingredient-containing coatings on carrier particles as so-called solid solutions comprising sparingly soluble substances. Solid solutions refer in accordance with the invention to systems in which no crystalline components of the sparingly soluble substance are observed.

The formulations obtained by the process according to the invention are, as stated, present in amorphous form which means that the crystalline components of the biologically active substance are less than 5% by weight. The amorphous state is preferably checked by means of DSC or XRD. Such an amorphous state can also be referred to as an X-ray amorphous state.

The process according to the invention allows the production of stable formulations with a high active ingredient loading and good stability with regard to the amorphous state of the sparingly soluble substance.

The special feature of this process is that an amphiphilic polymer is used together with sparingly soluble active ingredient in the form of a solvent-free melt. The amphiphilic polymer is capable of melting or dissolving the active ingredient even below its melting point. The coating is thus not a customary pellet or tablet coating, but rather a coating which keeps sparingly soluble active ingredient in dissolved form. The solid solution formed can also serve as a binder bridge between carrier particles and hence as a granulating aid. The advantage of solid solutions is that sparingly soluble active ingredients can better be made bioavailable thereby. The active ingredients may be present in the coating or binder layer in amorphous form or in molecularly dissolved form.

Owing to the amphiphilic polymer structure, the polymer is outstandingly suitable as a base for solid solutions. Solid dissolutions can, as already described, be achieved by means of a melt extrusion process. An elegant alternative process is the formation of the solid solution as a coating on solid dosage forms, such as pellets, granules, powders or else tablets. The use of common fluidized bed systems makes this process all the more interesting. Moreover, the freedom from solvent in the melt has a positive effect on the properties of the coatings. They are less porous, but smoother and more homogeneous.

A further advantage of this processing method is that solid solutions of sparingly soluble active ingredients can be processed to multiparticulate solid drug forms. These multiparticulate drug forms can be filled, for example, into hard gelatine capsules or even pressed to tablets.

### EXAMPLES

The amphiphilic copolymer used was a graft polymer formed from 13% by weight of polyethylene glycol, MW 6000, 57% by weight of N-vinylcaprolactam and 30% by weight of vinyl acetate with a mean molar mass of 44 000 daltons.

The processing was effected in a Glatt fluidized bed granulator (GPCG 3.1). The spraying was effected with a two-substance nozzle, diameter 4 mm.

The melt was obtained by heating the coating components to 70° C. and applying them to the carrier particles composed of sucrose (pellets with particle sizes of 710-850 μm (screen fraction)) or Granulac 200 (Lactose, from Meggle, (d50)=30 μm, bulk density 535 g/l).

The process parameters for processing in the fluidized bed granulator are specified for the individual examples.

The coatings produced were analyzed by means of XRD and DSC for crystallinity or amorphicity using the following instruments and conditions:

XRD (X-Ray Diffractogram)

Instrument: D 8 Advance diffractometer with 9-position sample changer (from Bruker/AXS)

Measurement method: θ-θ geometry in reflection

Angle range 2 theta: 2-80°

Step width: 0.02°

Measurement time per angle step: 4.8 s

Divergence slit: Göbel mirror with 0.4 mm inserted aperture

Antiscattering slit: Soller slit

Detector: Sol-X detector

Temperature: Room temperature

Generator setting: 40 kV/50 mA

In the examples which follow, different carrier particles were provided with active ingredient-containing coatings.

### Example 1

| Composition | Amount |
|---|---|
| PEG 6000 | 400 g |
| Copolymer | 50 g |
| Fenofibrate | 100 g |
| Sucrose pellets | 1000 g |
| Process parameter | Values |
| Feed air temperature [° C.] | 40 |
| Spray air pressure [bar] | 4.0 |

The XRD analysis did not show any crystalline active ingredient fractions.

The release of the active ingredient from 400 mg pellets was carried out in a USP apparatus 2 in 700 ml of 0.1 normal HCl. After 60 minutes, 90% of the active ingredient had been released.

### Example 2

| Composition | Amount |
|---|---|
| PEG 10 000 | 1600 g |
| PEG 400 | 50 g |
| Copolymer | 200 g |
| Cinnarizine | 100 g |
| Sucrose pellets | 1000 g |
| Process parameter | Values |
| Feed air temperature [° C.] | 45 |
| Spray air pressure [bar] | 4.0 |

The XRD analysis did not show any crystalline active ingredient fractions.

The release of the active ingredient from 0.6 g pellets was carried out in a USP apparatus 2 in 700 ml of 0.1 normal HCl. After 60 minutes, 70% of the active ingredient had been released.

### Example 3

| Composition | Amount |
|---|---|
| Lutrol F 68 ¹⁾ | 1400 g |
| Copolymer | 150 g |
| Piroxicam | 80 g |
| MCC pellets | 1000 g |
| Process parameter | Values |
| Feed air temperature [° C.] | 42 |
| Spray air pressure [bar] | 4.5 |

¹⁾ Poloxamer 188

The XRD analysis did not show any crystalline active ingredient fractions.

The release of the active ingredient from 400 mg pellets was carried out in a USP apparatus 2 in 700 ml of 0.1 normal HCl. After 40 minutes, 100% of the active ingredient had been released.

### Example 4

| Composition | Amount |
|---|---|
| Lutrol F 68 | 800 g |
| Lutrol F 127 ²⁾ | 1000 g |
| Copolymer | 200 g |
| Cinnarizine | 100 g |
| Sucrose pellets | 1000 g |
| Process parameter | Values |
| Feed air temperature [° C.] | 45 |
| Spray air pressure [bar] | 5 |

²⁾ Poloxamer 407

The XRD analysis did not show any crystalline active ingredient fractions.

The release of the active ingredient from 515 mg pellets was carried out in a USP apparatus 2 in 700 ml of 0.1 normal HCl. After 30 minutes, 100% of the active ingredient had been released.

### Example 5

| Composition | Amount |
|---|---|
| PEG 10 000 | 1600 g |
| Lutrol F 68 | 50 g |
| Copolymer | 200 g |
| Clotrimazole | 100 g |
| Sucrose pellets | 1000 g |
| Process parameter | Values |
| Feed air temperature [° C.] | 45 |
| Spray air pressure [bar] | 4.0 |

The XRD analysis did not show any crystalline active ingredient fractions.

The release of the active ingredient from 300 mg pellets was carried out in a USP apparatus 2 in 700 ml of 0.1 normal HCl. After 30 minutes, 100% of the active ingredient had been released.

### Example 6

| Composition | Amount |
|---|---|
| PEG 6000 | 2800 g |
| Copolymer | 160 g |
| Ketoconazole | 80 g |
| Sucrose pellets | 1000 g |
| Process parameter | Values |
| Feed air temperature [° C.] | 50 |
| Spray air pressure [bar] | 4.0 |

The XRD analysis did not show any crystalline active ingredient fractions.

The release of the active ingredient from 420 mg pellets was carried out in a USP apparatus 2 in 700 ml of 0.1 normal HCl. After 60 minutes, 100% of the active ingredient had been released.

### Example 7

| Composition | Amount |
|---|---|
| Lutrol F 127 | 1000 g |
| Lutrol F 68 | 1000 g |
| Copolymer | 220 g |
| Ketoconazole | 80 g |
| Sucrose pellets | 1000 g |
| Process parameter | Values |
| Feed air temperature [° C.] | 45 |
| Spray air pressure [bar] | 4.5 |

The XRD analysis did not show any crystalline active ingredient fractions.

The release of the active ingredient from 600 mg pellets was carried out in a USP apparatus 2 in 700 ml of 0.1 normal HCl. After 60 minutes, 100% of the active ingredient had been released.

### Example 8

| Composition | Amount |
|---|---|
| Lutrol F 127 | 1000 g |
| Lutrol F 68 | 1000 g |
| Copolymer | 220 g |
| Ketoconazole | 80 g |
| Granulac 200 | 1000 g |
| Process parameter | Values |
| Feed air temperature [° C.] | 45 |
| Spray air pressure [bar] | 4.5 |

The XRD analysis did not show any crystalline active ingredient fractions.

The release of the active ingredient from 600 mg granules was carried out in a USP apparatus 2 in 700 ml of 0.1 normal HCl. After 60 minutes, 100% of the active ingredient had been released.

### Example 9

| Substance | Amount |
|---|---|
| PEG 6000 | 1000 g |
| Copolymer | 220 g |
| Cinnarizine | 80 g |
| Granulac 200 | 1000 g |
| Process parameter | Values |
| Feed air temperature [° C.] | 45 |
| Spray air pressure [bar] | 4.5 |

The XRD analysis did not show any crystalline active ingredient fractions.

The release of the active ingredient from 400 mg granules was carried out in a USP apparatus 2 in 700 ml of 0.1 normal HCl. After 60 minutes, 85% of the active ingredient had been released.

What is claimed is:

1. A formulation of one or more sparingly water-soluble active ingredients comprising carrier particles provided with active ingredient-containing coatings, the one or more sparingly water-soluble active ingredients being embedded in coatings composed of amphiphilic copolymers, and the coatings being applied in the form of a solvent-free melt.

2. The formulation according to claim 1, wherein the amphiphilic copolymers are obtained by free-radically initiated polymerization of a mixture of
i) 30 to 80% by weight of N-vinyllactam,
ii) 10 to 50% by weight of vinyl acetate and
iii) 10 to 50% by weight of a polyether,
with the proviso that the sum of i), ii) and iii) is 100% by weight.

3. The formulation according to claim 2, wherein the amphiphilic copolymers are obtained from:
i) 30 to 70% by weight of N-vinyllactam
ii) 15 to 35% by weight of vinyl acetate and
iii) 10 to 35% by weight of a polyether.

4. The formulation according to claim 3, wherein the amphiphilic copolymers are obtained from:
i) 40 to 60% by weight of N-vinyllactam
ii) 15 to 35% by weight of vinyl acetate and
iii) 10 to 30% by weight of a polyether.

5. The formulation according to claim 4, wherein the amphiphilic copolymers are obtained from:
i) 50 to 60% by weight of N-vinyllactam
ii) 25 to 35% by weight of vinyl acetate and
iii) 10 to 20% by weight of a polyether.

6. The formulation according to claim 1, wherein the carrier particles are pellets composed of pharmaceutical excipients.

7. The formulation according to claim 1, wherein the carrier particles comprise sucrose, carrageenan, starch, lactose or microcrystalline cellulose.

8. The formulation according to claim 1, wherein the carrier particles have particle sizes in the range of of 100 to 2000 μm.

9. The formulation according to claim 1, wherein the carrier particles comprise an active ingredient and an amphiphilic polymer.

10. The formulation according to claim 1, wherein the coatings additionally comprise pharmaceutical excipients.

11. The formulation according to claim 1, wherein the coatings comprise 20 to 99% by weight of the amphiphilic copolymers.

12. A process for producing the formulation according to claim 1, which comprises producing the formulations by applying a melt comprising one or more sparingly water-soluble active ingredients and an amphiphilic copolymer onto a fluidized bed composed of carrier particles.

13. The process according to claim 12, wherein the melt is applied by spraying.

14. The process according to claim 12, wherein the melt is obtained at temperatures of 30 to 260° C.

15. The process according to claim 12, wherein the melt is solvent-free.

16. The process according to claim 12, wherein the solvent content of the melt is less than 5000 ppm.

17. A dosage form comprising the formulation according to claim 1.

18. The dosage form according to claim 17 in the form of tablets, capsules or sachets.

19. The formulation of claim 1, wherein the ampiphilic copolymers are obtained from a mixture of an N-vinyllactam selected from the group consisting of N-vinylcaprolactam or N-vinylpyrrolidone, and mixtures thereof; vinyl acetate; and a polyether selected from the group consisting of: polyethylene glycol, polypropylene glycol, polytetrahydrofurans, polybutylene glycol obtained from 2-ethyloxirane or 2,3-dimethyloxirane, and mixtures thereof.

20. The formulation of claim 1, wherein the one or more sparingly water-soluble active ingredients is selected from the group consisting of benzodiazepines, antihypertensives, vitamins, cytostatics, anesthetics, neuroleptics, antidepressives, antivirals, antibiotics, antimycotics, antidementives, fungicides, chemotherapeutics, urologics, thrombocyte aggregation inhibitors, sulfonamides, spasmolytics, hormones, immunoglobulins, sera, thyroid therapeutics, psychopharmaceuticals, Parkinson's drugs, ophthalmics, neuropathy preparations, calcium metabolism regulators, muscle relaxants, anesthetics, lipid-lowering drugs, liver therapeutics, coronary drugs, cardiac drugs, immunotherapeutics, regulatory peptides and inhibitors thereof, hypnotics, sedatives, gynaecologicals, gout remedies, fibrinolytics, enzyme preparations, transport proteins, enzyme inhibitors, emetics, blood-flow stimulators, diuretics, diagnostics, corticoids, cholinergics, biliary therapeutics, antiasthmatics, bronchodilators, beta-receptor blockers, calcium antagonists, ACE inhibitors, arteriosclerotic drugs, anti-inflammation drugs, anticoagulants, antihypotensives, antihypoglycemics, antihypertensives, antifibrinolytics, antiepileptics, antiemetics, antidotes, antidiabetics, antiarrhythmics, antianemics, antiallergics, anthelmintics, analgesics, analeptics, aldosterone antagonists, and slimming drugs.
